SEQUENCE LISTING (2 sequences)

SEQ ID NO: 1 - DNA, 40 bp, Artificial sequence
  oligonucleotide 1
  misc_feature (26)..(26): deoxythymidine nucleotide (T) labelled with Atto 655 dye
  misc_feature (27)..(27): deoxythymidine nucleotide (T) labelled with Atto 655 dye
  misc_feature (28)..(28): deoxythymidine nucleotide labelled with a BHQ-2 quencher
  misc_feature (29)..(29): deoxythymidine nucleotide (T) labelled with Atto 655 dye
  misc_feature (30)..(30): deoxythymidine nucleotide (T) labelled with Atto 655 dye
tcgtgcctcatcgaacatgacgaggtttttggtttgtggt

SEQ ID NO: 2 - DNA, 115 bp, Artificial sequence
  oligonucleotide 2
  misc_feature (1)..(1): 5′ phosphate group attached to cytosine
catgttcgatgaggcacgatagatgtacgctttgacatacgctttgacaatacttgagcagtcggcagatataggatgttgcaagctccgtgagtcccacaaaccaaaaacctcg

(19) United States
(12) Patent Application Publication
(10) Pub. No.: US 2018/0008985 A1
(43) Pub. Date: Jan. 11, 2018

(54) IMPROVED DROPLET SEQUENCING APPARATUS AND METHOD

(71) Applicant: BASE4 INNOVATION LTD, Cambridge, Cambridgeshire (GB)

(72) Inventors: Barnaby BALMFORTH, Cambridgeshire (GB); Cameron Alexander FRAYLING, Cambridgeshire (GB); Thomas Henry ISAAC, Cambridgeshire (GB)

(73) Assignee: BASE4 INNOVATION LTD, Cambridge, Cambridgeshire (GB)

(21) Appl. No.: 15/543,780

(22) PCT Filed: Jan. 21, 2016
(86) PCT No.: PCT/GB2016/050130

§ 371 Date: Jul. 14, 2017

(30) Foreign Application Priority Data
Jan. 21, 2015 (GB) 1501012.7

(51) Int. Cl. B01L 3/00; B01F 3/08; B01L 3/02

(52) U.S. Cl. CPC B01L 3/502784; B01L 3/0268; B01L 3/5088; B01F 3/0807; C12Q 2563/159; C12Q 2565/629; B01L 2300/0819

(57) ABSTRACT

An apparatus for sequencing a polynucleotide analyte is provided and comprises; •a first zone in which a stream of single nucleotides is generated by progressive digestion of a molecule of the analyte attached to a particle located therein and exposed to a flowing aqueous medium; •a second zone in which a corresponding stream of aqueous droplets is generated from the aqueous medium and the nucleotide stream and wherein at least some of the droplets contain a single nucleotide and •a third zone in which each droplet is stored and/or interrogated to reveal a property characteristic of the single nucleotide it may contain; characterised in that the first zone comprises a microfluidic channel through which the aqueous medium flows and the location comprises a hollow seating in a wall thereof to which suction can be applied and into which the particle can be close-fitted.

This invention relates to an improved apparatus and method for sequencing polynucleotides such as RNA and DNA fragments derived for example from genetic material of natural origin or synthetic analogues thereof.

Next generation sequencing of genetic material is already making a significant impact on the biological sciences in general and medicine in particular as the unit cost of sequencing falls in line with the coming to market of faster and faster sequencing machines. In our previous applications WO 2014/053853 and WO 2014/053854 we have described a new sequencing method and associated apparatus which involves progressive digestion of a polynucleotide analyte to generate a stream of single-nucleobase nucleotides (hereinafter ‘single nucleotides’), preferably an ordered stream thereof generated by pyrophosphorolysis, each of which can be captured one-by-one into corresponding droplets in a microdroplet stream. Thereafter, each droplet can be chemically and/or enzymatically manipulated to reveal the particular single nucleotide it originally contained. In one embodiment, these chemical and/or enzymatic manipulations comprise a method involving the use of one or more two-component oligonucleotide probe types each of which is adapted to be able to selectively capture one of the single nucleotide types from which the analyte is constituted; for example in the case of naturally occurring DNA the one of the four types corresponding to the nucleobases cytosine, thymine, guanine and adenine. Typically, in each such probe type, one of the two oligonucleotide components comprises distinct and characteristic fluorophores and in the probe's unused state the ability of these fluorophores to fluoresce remains extinguished by virtue of either the presence of quenchers located close-by or by self-quenching. Thereafter, once a particular probe has captured its corresponding single nucleotide, it becomes susceptible to exonucleolysis thereby causing the fluorophores to become separated from the quenchers and/or each other and thus enabling them to fluoresce freely at their characteristic wavelength. By this means, the nature of the single nucleotide originally present in each droplet can be inferred from spectroscopic analysis.

J. Biotech. 102 (2003), pp. 1-14, U52008/0194012 and Lab on a Chip (2012), pp. 906-915 teach vacuum immobilisation of beads for various different purposes.

We have now developed improved versions of an apparatus for use with sequencing methods of this type which allow the initial polynucleotide digestion step to be easily and reliably conducted and in a way which can be multiplexed to work with many thousands of droplet generation sites. The improvement involves first immobilising the polynucleotide to be analysed (hereinafter the analyte) on the surface of a particle and thereafter immobilising the particle at a location within the apparatus where digestion is to occur by the application of a negative pressure gradient across it. We are aware that U.S. Pat. No. 6,471,917 has previously described systems and techniques for placing small solid beads into an organized array but to the best of our knowledge such technologies have not been applied to holding single molecules under flow or to single molecule sequencing applications.

Thus, according to a first aspect of the invention there is provided an apparatus for sequencing a polynucleotide analyte comprising;
a first zone in which a stream of single nucleotides is generated by progressive digestion of a molecule of the analyte attached to a particle located therein and exposed to a flowing aqueous medium;
a second zone in which a corresponding stream of aqueous droplets is generated from the aqueous medium and the nucleotide stream and wherein at least some of the droplets contain a single nucleotide and
a third zone in which each droplet is stored and/or interrogated to reveal a property characteristic of the single nucleotide it may contain;
   characterised in that the first zone comprises a microfluidic channel through which the aqueous medium flows and the location comprises a hollow seating in a wall thereof to which suction can be applied and into which the particle can be close-fitted.

The apparatus of the present invention involves zones in which a stream, preferably an ordered stream, of single nucleotides is generated by progressive digestion of a polynucleotide analyte using an enzyme. In one embodiment this progressive digestion comprises exonucleolysis using an exonuclease. In another it comprises phosphorolysis using a phosphorylase. In yet another, the digestion comprises pyrophosphorolysis using a polymerase Depending on the particular mode of digestion chosen the analyte may be a single-stranded, a double-stranded or a partially double-stranded polynucleotide and may have a nucleotide chain length which can in principle be unlimited; for example up to and including the many millions of nucleotides or nucleotide pairs found in a fragment of a genome. In one embodiment, the analyte will therefore be at least 50, preferably at least 150 nucleotides or nucleotide pairs long; suitably it will be greater than 500, greater than 1000 and in some cases 5000+ nucleotide pairs long. The analyte itself is suitably comprised of RNA or DNA of natural origin (e.g. genetic material derived from a plant, animal, bacterium or a virus) although the method is equally applicable to the sequencing of partially or wholly synthetic RNA or DNA or indeed other nucleic acids made up wholly or in part of nucleotides comprised of nucleotide bases that are not commonly encountered in nature; i.e. nucleotides having nucleobases other than adenine, thymine, guanine, cytosine and uracil. Examples of such nucleobases include 4-acetylcytidine, 5-(carboxyhydroxylmethyl)uridine, 2-O-methylcytidine, 5-carboxymethylaminomethyl-2-thiouridine, 5-carboxymethylamino-methyluridine, dihydrouridine, 2-O-methylpseudouridine, 2-O-methylguanosine, inosine, N6-isopentyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, 5-methoxyuridine, 5-methoxycarbonylmethyl-2-thiouridine, 5-methoxycarbonylmethyluridine, 2-methylthio-N6-isopentenyladenosine, uridine-5-oxyacetic acid-methylester, uridine-5-oxyacetic acid, wybutoxosine, wybutosine, pseudouridine, queuosine, 2-thiocytidine, 5-methyl-2-thiouridine, 2-thiouridine, 4-thiouridine, 5-methyluridine, 2-O-methyl-5-methyluridine and 2-O-methyluridine. In the case of exonucleolysis, phosphorolysis and pyrophosphorolysis the single nucleotides produced are respectively nucleoside monophosphate, nucleoside diphosphates and nucleoside triphosphates with their exact form depending on whether the analyte is DNA or RNA.

In one version of the apparatus, the analyte is progressively pyrophosphorolysed in the 3′-5′ direction to generate a stream of single nucleotide triphosphates the order of which corresponds to that of the sequence of the analyte. The pyrophosphorolysis itself is generally carried out at a temperature in the range 20 to 90° C. in the presence of a reaction medium including a polymerase. Suitably it is also carried out so that the single nucleotides are continually removed from the region of pyrophosphorolysis around the particle by a flowing aqueous medium. This removal may occur at or immediately adjacent to an outlet at which the droplet is produced. In one embodiment this medium is buffered and also contains those other components needed in order to sustain the pyrophosphorolysis reaction (polymerase, pyrophosphate anion, magnesium cation etc.). In another, it additionally contains one or more of (a) the probe types specified below (or probes having an equivalent function); (b) the various chemicals and enzymes required to cause the probe to bind to and capture the relevant single nucleotide (e.g. polymerase and/or ligase) and (3) the enzyme required to cause subsequent exonucleolysis of the used probe to occur. Alternatively, some or all of these components may be introduced together or in stages into the flowing aqueous medium or the droplets (as the case may be) at some other point(s) upstream of the third zone. Such subsequent introduction of these components directly into the droplets can be achieved for example by injection using an injector or by droplet coalescence.

Preferably the apparatus is designed so that the rate of pyrophosphorolysis is as fast as possible and in one embodiment this rate lies in the range from 1 to 50 single nucleotides per second. Further information about the pyrophosphorolysis reaction as applied to the progressive degradation of polynucleotides can be found for example in J. Biol. Chem. 244 (1969) pp. 3019-3028.

In the apparatus of the invention, the analyte molecule being subjected to digestion is suitably pre-attached to a particle. In one embodiment this particle comprises a bead; for example a microbead, made of an inert material such as glass, silica, alumina, a metal or a non-degradable polymer. In another embodiment, the bead is made of a magnetic material enabling it to be introduced into the apparatus and delivered to the desired location magnetically.

Preferably, the particle further comprises an outer surface specifically adapted to bind physically or chemically to the analyte. There are many ways in which the analyte can be so bound any of which can in principle be used. For example, one approach involves priming the surface of a glass bead with a functionalised silane such as an epoxysilane, an aminohydrocarbylsilane or a mercaptosilane. The reactive sites so generated can then be treated with a derivative of the analyte which has been correspondingly modified to include a terminal amine, succinyl or thiol group. In one embodiment chemical attachment can take place via ligation to adaptor oligonucleotides or via biotin-streptavidin conjugation.

In another embodiment the particles so primed will have only one reactive site so that only one molecule of the analyte can be attached thereto. This is important if the analyte is a small polynucleotide fragment since otherwise multiple molecules tend to become attached which is undesirable. It is of a lesser concern if the fragment is large since steric effects will work to militate against such a phenomenon taking place. Suitably, the particle will have a maximum diameter in the range 1 to 5 microns.

The first zone employed in the apparatus is suitably of microfluidic dimensions and includes at least one location where the particle can be positioned and held immobile by the application of a negative pressure gradient. In one embodiment the first zone includes a microfluidic channel such as a micron-sized chamber or a piece of microfluidic tubing, and the location comprises a hollow seating, e.g. a frustoconical seating, in a wall of the chamber or tubing into which the particle can be close-fitted. In this arrangement, the hollow interior of the seating is connected to the inlet of a second channel to which vacuum suction can be applied. Once the particle is in place, a negative pressure gradient can thereafter be maintained by, for example, applying continuous suction or by evacuating the interior of the hollow seating and sealing it under static vacuum. In this apparatus type the hollow seating is suitably located immediately upstream of the second zone.

The aqueous medium issues from the first zone in the form of a liquid stream containing the single nucleotides separated spatially and temporally from each other and arranged in an order corresponding to that of the nucleotide sequence of the analyte. Thereafter, in the second zone this aqueous stream is then converted into a corresponding stream of aqueous droplets, suitably microdroplets, at least some of which contain a single nucleotide, by causing the aqueous medium to issue forth from a droplet-generating head of suitable dimensions and geometry into a flowing carrier medium comprising an immiscible solvent such as a mineral or hydrocarbon oil; for example silicone oil. Alternatively, and in a preferred embodiment, the particle may be immobilised in the first zone upstream of a printer nozzle or like component in which case optionally the carrier medium concerned may be dispensed with or rendered non-flowing. To avoid the risk that a given microdroplet contains more than one single nucleotide, it is preferred to release the single nucleotide in the pyrophosphorolysis step and/or to adjust the flow of the aqueous medium through the first zone so that each filled microdroplet generated by the nozzle is separated by from 1 to 20 preferably 2 to 10 empty ones. Thereafter, the stream of filled and unfilled microdroplets, optionally in the solvent, are either printed onto or caused to flow along a flow path, suitably a microfluidic flow path, to the third zone at a rate and in a manner such that the microdroplets are maintained in a discrete state and do not have the opportunity to coalesce with each other. Suitably the microdroplets employed have a diameter less than 100 microns, preferably less than 50 microns, more preferably less than 20 microns and even more preferably less than 15 microns. Most preferably of all, their diameters are in the range 2 to 20 microns. In one embodiment, the microdroplet flow rate from the second to third zones is in the range 50 to 3000 droplets per second preferably 100 to 2000.

The third zone in fluid connection with the second zone is adapted to receive and store the droplets. In one embodiment, this third zone comprises a surface, such as that of a metal or plastic sheet or glass slide which is moveable relative to the egress point of the second zone (e.g. the printer nozzle) in effect enabling each droplet to be printed onto the surface at a specified point in a repeatable manner akin to that of an ink-jet printer. In one embodiment of this design, the receiving surface is patterned with an array of discrete droplet-receiving locations, such as wells or droplet binding sites, to which each droplet can be delivered and immobilised. In another embodiment the surface is pre-covered with a droplet-immiscible liquid layer which assists in preventing adjacent droplets undergoing migration and coalescence. In a preferred version of this design this liquid layer comprises a UV-light curable monomer so that once all the droplet-receiving locations have been filled, the liquid can be cured and the droplets may be permanently encapsulated in a transparent, solid polymer matrix.

Thereafter the patterned surface is interrogated in turn at each droplet-receiving location to reveal a property characteristic of the single nucleotide that each droplet may contain. Although this interrogation can be made separate from the apparatus, for example optionally within another specialised device such as a spectrometer, it is preferably carried out using an interrogation means which is an integral part of the third zone.

The property to be detected by the interrogation means can in principle be any characteristic property of the single nucleotide which is made manifest directly or indirectly. For example, in one embodiment, the nature of each single nucleoside monophosphate, diphosphate or triphosphate, as the case may be, is determined directly by interrogating each droplet with electromagnetic radiation and studying the light which undergoes Raman-scattering. In this embodiment, the Raman-scattering originating from the single nucleotide is preferably enhanced by including within the droplet a colloid of a metal, such as gold or silver, capable of being stimulated to undergo plasmonic resonance. In another, preferred embodiment, fluorescence radiation arising from fluorophores present in the droplets is measured. In both these embodiments, the interrogation means suitably comprises a source of focused incident light, for example a laser beam; a photodetector or like device tuned to a characteristic wavelength(s) or wavelength envelope(s) of either a vibrational mode of the nucleotide or a florescence mode of the fluorophore; a means to move the source of incident light, the photodetector and the droplets on the surface relative to one another (e.g. a mechanical stage) and associated optics for directing and focusing beams of the incident and fluorescent light.

Both apparatus types may also further comprise a microprocessor integral therewith for analysing data generated by the interrogation means which is characteristic of the sequence of the analyte. This duty can alternatively be performed remotely using a stand-alone computer if so desired.

The apparatus is especially useful for creating and interrogating droplets in which the single nucleotides they contain have been previously captured selectively by probes which are capable of fluorescing substantially only after they have been used and undergone subsequent exonucleolysis. In general terms, this is achieved by ensuring that the apparatus is capable of processing droplets which at some point contains molecules of at least one probe type selective for one of the various single nucleotides from which the analyte is constructed and enzymes so as to allow incorporation of the single nucleotide into its corresponding probe; using e.g. polymerase and/or ligase. Each droplet will also contain, at some point, a 3′-5′ exonuclease or a polymerase demonstrating an equivalent activity; a pyrophosphatase, buffer, surfactants and other components conventional in the biochemical art. In one embodiment, some, all or any permutation of these probes, enzymes, exonuclease and other components are introduced into the original aqueous medium in the first zone or contained within the original aqueous medium feed itself. In another some, all or any permutation of these probes, enzymes, exonuclease and other components are introduced into the aqueous medium as or after each droplet is created. In this case these material may introduced into the droplet by for example direct injection or by locating them first in secondary droplets comprising a secondary droplet stream and thereafter merging droplets from that stream one-by-one with corresponding droplets in the primary stream.

As regards the probes themselves, one class which may advantageously be used is that based on the various capture systems and their modes of utilisation taught in our patent applications WO 2014/053853, WO 2014/053854, WO 2014/167323, and WO 2014/167324; the contents of which are incorporated herein by reference. Briefly, one sub-class of these probes, in their unused state, is characterised by comprising a single-stranded nucleotide region the ends of which are each attached to two different double-stranded oligonucleotide regions. Preferably, at least one of the oligonucleotide regions comprises multiple fluorophores exhibiting characteristic fluorescence and optionally quenchers. The fluorophores are arranged on the oligonucleotide region so that that the fluorescence they exhibit is substantially less than when the same number of fluorophores is bound to a corresponding number of single nucleotides. Preferably the probes in their unused state do exhibit either no or substantially no fluorescence at all. These probes work by capturing their corresponding single nucleotide using a polymerase and optionally a ligase to generate a used probe which is then susceptible to exonucleolysis leading to the generation of multiple single nucleotides bearing the fluorophores which are then able to fluoresce fully. In another preferred sub-class, these probes are composed of two components; complementary i- and j-shaped oligonucleotides, either or both of which can be labelled, which can be hybridised together in the presence of their target single nucleotide into a substantially double-stranded oligonucleotide which can also be digested by an exonuclease as explained above

In yet another preferred sub-class, disclosed in our pending application PCT/GB2015/052119 and also incorporated herein by reference, the probe comprises (a) a first single-stranded oligonucleotide labelled with characteristic fluorophores in an undetectable state and (b) second and third single-stranded oligonucleotides capable of hybridising to complementary regions on the first oligonucleotide. In the case of this particular sub-class the used probe can be digested with an enzyme having double-stranded exonucleolytic activity to yield the detectable elements in a detectable state and a single-stranded fourth oligonucleotide which is at least in part the sequence complement of the first oligonucleotide. As a consequence, the fourth oligonucleotide can be caused to react with another labelled first oligonucleotide to produce a substantially double-stranded oligonucleotide product again corresponding to the used probe. By repeating both this and the exonucleolysis step cyclically, the number of free fluorophores capable of fluorescing can be caused to cascade and increase significantly. Most preferred items in the class comprise those probes in which the second and third oligonucleotides comprise a single oligonucleotide regions of a common fourth oligonucleotide which upon capturing its complementary target single nucleotide generates a single-stranded closed-loop oligonucleotide resistant to exonucleolysis and which can act as a template for hybridisation and subsequent exonucleolysis of a series of first oligonucleotides.

It will be apparent from the above that the apparatus of the present invention is in one embodiment preferably designed to be used with particular methods for generating fluorescence in droplets containing single nucleotides. Thus according to a second aspect of the invention there is provided further method of sequencing a polynucleotide analyte including the steps of:
(a) generating a stream of single nucleotide triphosphates by progressive pyrophosphorolysis of an analyte molecule attached to a particle exposed to a flowing aqueous medium;
(b) generating a stream of droplets from the aqueous medium and the stream of single nucleotides wherein at least some of the droplets contain a single nucleotide and
(c) storing and/or interrogating each droplet and detecting a property characteristic of the single nucleotide it may contain;
   characterised in that step (a) further includes the sub-step of immobilising the particle in a close fitting, hollow seating in a microfluidic channel to which suction can be applied.

In one embodiment of the method, the aqueous medium contains at a given point at least one single-nucleotide probe selective for capturing one of the nucleotide types from which the analyte is constituted and the said probe(s) are capable of fluorescing substantially only after they have captured a single nucleotide and undergone subsequent exonucleolysis. Preferably these probes are members of the classes described above. In another embodiment, the probe(s) are either contained within the original flowing aqueous medium or subsequently introduced directly into each droplet after it has been created. In yet another, the methods further comprise step (d) which comprises detecting the fluorescence radiation emitted by each droplet and assembling sequence data therefrom.

In another embodiment the method includes the pre-step of attaching analyte molecules to the surfaces of a plurality of particles; selecting a particle for use and attaching the chosen particle to its location by means of suction. The particles here suitably comprise beads having reactive surfaces.

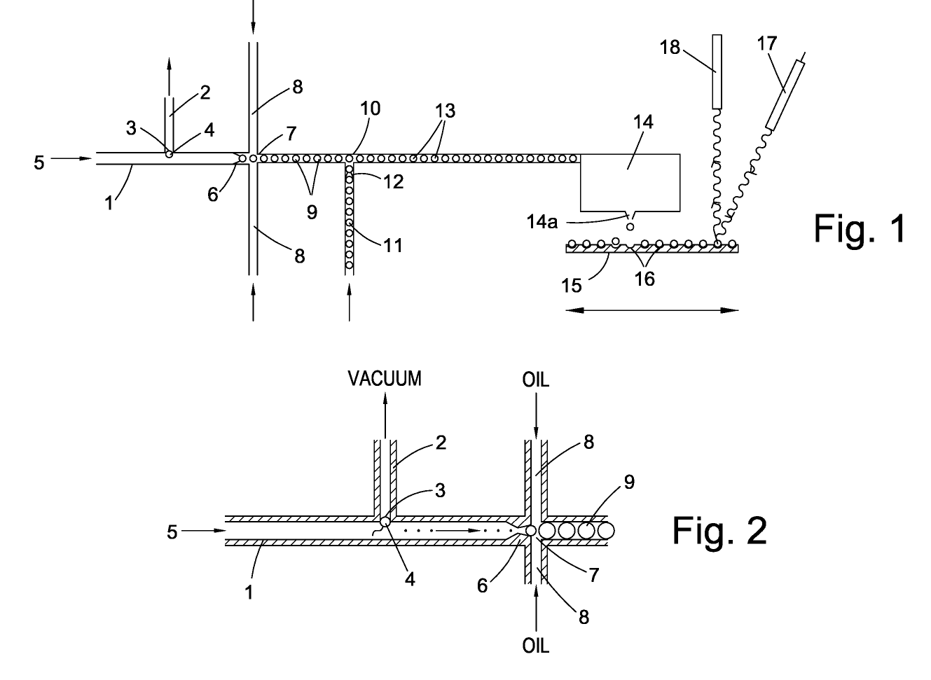

An apparatus according to the invention is now illustrated by the following FIGS. 1 and 2 which schematically illustrate a sequencing device according to the present invention in which aqueous droplets containing a single nucleotide and nucleotide-detecting oligonucleotide probe system are created and interrogated.

A ten micron diameter microfluidic tube 1 is provided with a side-channel 2 to which vacuum suction can be applied. 2 intersects 1 at a junction comprising a frustoconical orifice 3 of circular cross-section with an internal diameter of two microns. A spherical glass bead 4 of diameter four microns is previously introduced into 1 and seated in 3 so that a significant part of its outer surface protrudes into a stream of aqueous medium 5 when the latter is caused to flow through 1 from a point of introduction upstream of 3. Attached to 4 by streptavidin conjugation is an analyte comprising 1000 nucleobase double-stranded fragment of DNA. 5 comprises a buffered (pH 7.5) reaction medium at 37° C., comprising Bst Large Fragment DNA Polymerase and a 2 millimoles per litre concentration of each of sodium pyrophosphate and magnesium chloride. The temperature of the bead is maintained at 37° C. and under these conditions the polymerase progressively digests the analyte in the 3′ to 5′ direction thereby releasing a stream of single nucleotides (deoxyribonucleotide triphosphates) which are then carried downstream of 4 by the flow of the medium. The order of single nucleotides in the downstream part of 5 thus corresponds to their original sequence in the analyte. Downstream part of 5 emerges from a droplet head 6 into a first chamber 7 where it is contacted with one or more streams of light silicone oil 8. The velocities of these streams at the point of contact are chosen to avoid turbulent mixing and to create substantially aqueous spherical droplets 9 suspended in the oil each having a diameter of approximately eight microns. A stream of 9 is then carried forward along a second microfluidic tube of the same diameter at a rate of 1000 droplets per second to a second chamber 10 into which a second stream of five micron aqueous spherical droplets 11 is also fed using a second droplet head 12. Droplets 11 comprise a nucleotide-detecting oligonucleotide probe system such as that outlined below and are caused to coalesce in a sequential fashion with 9 to form enlarged aqueous droplets 13 approximately nine microns in diameter. The stream of droplets 13 so created is then delivered to a printer assembly 14 provided with a droplet printer head 14a where each droplet is in turn printed onto a glass slide 15 moveable along both axes of the plane which defines its face and patterned with a two-dimensional array of wells 16 for containing the individual droplets.

The droplets are then incubated on the glass slide before being interrogated by a detection system comprising one or more lasers 17 and detectors 18 tuned to the appropriate wavelengths for the excitation and detection of fluorescence from the fluorescent dyes used in the probe system. The detection of fluorescence at a given wavelength above a determined threshold then indicates the presence of a single nucleotide base of a given type within the droplet. Thus as the droplets are interrogated in turn the sequence of nucleotide bases in the original polynucleotide analyte can in effect be read off.

An example of an oligonucleotide probe system that may be used in the system described above is now described in detail.

A single-stranded first oligonucleotide 1 is prepared, having the following nucleotide sequence:

5′TCGTGCCTCATCGAACATGACGAGGXXQXXGGTTTGTGGT3′

wherein A, C, G, and T represent nucleotides bearing the relevant characteristic nucleotide base of DNA; X represents a deoxythymidine nucleotide (T) labelled with Atto 655 dye using conventional amine attachment chemistry and Q represents a deoxythymidine nucleotide labelled with a BHQ-2 quencher. It further comprises a capture region (A nucleotide) selective for capturing deoxythymidine triphosphate nucleotides (dTTPs).

Another single-stranded oligonucleotide 2, comprising (1) a second oligonucleotide region having a sequence complementary to the 3′ end of the first oligonucleotide with a single base mismatch; (2) a third oligonucleotide region having a sequence complementary to the 5′ end of the first oligonucleotide and (3) a 76 base pair single-stranded linker region, is also prepared. It has the following nucleotide sequence:

5′PCATGTTCGATGAGGCACGATAGATGTACGCTTTGACATACGCTTTGA
CAATACTTGAGCAGTCGGCAGATATAGGATGTTGCAAGCTCCGTGAGTCC
CACAAACCAAAAACCTCG3′

wherein additionally P represents a 5′ phosphate group.

A reaction mixture comprising the probe system is then prepared having a composition corresponding to that derived from the following formulation:

56 uL 5× buffer pH 7.5

28 uL oligonucleotide 1, 100 nM

28 uL oligonucleotide 2, 10 nM

2.8 uL mixture of dNTPs (including dTTP), 10 nM

0.4 U Phusion II Hot Start polymerase (exonuclease)

1.6 U Bst Large Fragment polymerase

20 U E. coli ligase

4 U Thermostable Inorganic Pyrophosphatase

Water to 280 uL

wherein the 5× buffer comprises the following mixture:

200 uL Trizma hydrochloride, 1M, pH 7.5

13.75 uL aqueous MgCl₂, 1M

2.5 uL Dithiothreitol, 1M

50 uL Triton X-100 surfactant (10%)

20 uL Nicotinamide adenine dinucleotide, 100 uM

166.67 uL KCl

Water to 1 mL

In the presence of a single dTTP nucleotide, said nucleotide is incorporated onto the 3′ end of one of the oligonucleotides 2 and ligation of oligonucleotide 2 to form a closed-loop used probe occurs. This process is carried out by incubating the mixture at 37° C. for 50 minutes. The reaction medium is then incubated at 70° C. for a further 50 minutes, activating the Phusion II polymerase. One of the oligonucleotides 1 can anneal to a circularised oligonucleotide 2 at this temperature and in this double-stranded form is digested by the polymerase, releasing its fluorophores into a detectable state. A further oligonucleotide 1 is then able to anneal to the circularised probe, allowing this process to repeat in a continuous cycle and resulting in a growth of fluorescence intensity over time.

Further sets of similar oligonucleotide probes are also prepared having different capture sites, sequences and fluorophores which, combined with the first probe set, allow the capture, detection and discrimination of the four nucleotide bases to be achieved.

1. An apparatus for sequencing a polynucleotide analyte, said apparatus comprising:
a first zone in which a stream of single nucleotides is generated by progressive digestion of a molecule of the polynucleotide analyte attached to a particle located therein and exposed to a flowing aqueous medium;
a second zone in which a corresponding stream of aqueous droplets is generated from the aqueous medium and the nucleotide stream and wherein at least some of the droplets contain a single nucleotide; and
a third zone in which each droplet is stored and/or interrogated to reveal a property characteristic of the single nucleotide it may contain;
characterised in that the first zone comprises a microfluidic channel through which the aqueous medium flows, and a location comprising a hollow seating in a wall thereof to which suction can be applied and into which the particle can be close-fitted.

2. The apparatus as claimed in claim 1, characterised in that the hollow seating is located immediately upstream of the second zone.

3. The apparatus as claimed in claim 1, characterised in that the particle comprises a bead having a surface to which the analyte molecule can be physically or chemically bound.

4. The apparatus as claimed in claim 1, characterised in that the digestion method is selected from exonucleolysis, phosphorolysis or pyrophosphorolysis.

5. The apparatus as claimed in claim 1, characterised in that the third zone includes a laser and a photodetector to detect Raman-scattered light.

6. The apparatus as claimed in claim 1, characterised by being capable of processing an aqueous medium which in at least one of the second or third zones contains at least one single-nucleotide probe selective for one of the nucleobase types from which the analyte is constituted; said probe(s) being capable of fluorescing substantially only after it has captured a single nucleotide and undergone subsequent exonucleolysis.

7. The apparatus as claimed in claim 6, characterised by further comprising a means to introduce the probe(s) into the aqueous medium before, as or after each droplet is created.

8. The apparatus as claimed in claim 1, characterised in that the third zone includes a printer nozzle adapted to print each droplet onto a surface comprised of an array of droplet-receiving locations.

9. The apparatus as claimed in claim 1, characterised in that the third zone includes an interrogation means for detecting fluorescence radiation emitted from each droplet.

10. A method of sequencing a polynucleotide analyte including the steps of:
(a) generating a stream of single nucleotide triphosphates by progressive pyrophosphorolysis of an analyte molecule attached to a particle exposed to a flowing aqueous medium;
(b) generating a stream of droplets from the aqueous medium and the stream of the single nucleotides, wherein at least some of the droplets contain a single nucleotide, and
(c) storing and/or interrogating each droplet and detecting a property characteristic of the single nucleotide it may contain;
characterised in that step (a) further includes the sub-step of immobilising the particle in a close-fitting hollow seating in a microfluidic channel to which suction can be applied.

11. The method as claimed in claim 10, characterised in that the aqueous medium contains at a given point at least one single-nucleotide probe selective for capturing one of the nucleotide triphosphate types from which the polynucleotide analyte is constituted; said probe(s) being capable of fluorescing substantially only after they have captured a single nucleotide and undergone subsequent exonucleolysis.

12. The method as claimed in claim 11, characterised in that the probe(s) comprises (a) a first single-stranded oligonucleotide labelled with characteristic fluorophores in an undetectable state, and (b) second and third single-stranded oligonucleotides capable of hybridising to complementary regions on the first oligonucleotide.

13. The method as claimed in claim 12, characterised in that the second and third oligonucleotides are oligonucleotide regions of a single oligonucleotide so that addition of a target single nucleotide creates a single-stranded closed loop resistant to exonucleolysis.

14. The method as claimed in claim 11, characterised in that the probe(s) are either contained within the original flowing aqueous medium or subsequently introduced directly into each droplet after it has been created.

15. The method as claimed in claim 10, characterised in that the particle comprises a bead having a reactive surface onto which the analyte molecule is attached.
